(19) United States
(12) Patent Application Publication
(10) Pub. No.: US 2009/0215791 A1
(43) Pub. Date: Aug. 27, 2009

(54) NEW THERAPEUTIC USE OF 4-(2-FLUOROPHENYL)-6-METHYL-2-(1-PIPERAZINYL)THIENO[2,3-D]PYRIMIDINE

(75) Inventors: Hazel Judith Bardsley, Konstanz (DE); David Cavalla, Cambridge (GB); Robert William Gristwood, Cambridge (GB)

Correspondence Address: HAMILTON, BROOK, SMITH & REYNOLDS, P.C., 530 VIRGINIA ROAD, P.O. BOX 9133, CONCORD, MA, 01742-9133, US

(73) Assignee: Dynogen Pharmaceuticals, Inc., Waltham, MA (US)

(21) Appl. No.: 12/200,102

(22) Filed: Aug. 28, 2008

Related U.S. Application Data
(63) Continuation of application No. 10/617,847, filed on Jul. 10, 2003, now abandoned.
Application No. 10/617,847 is a continuation-in-part of application No. PCT/GB02/02388, filed on May 21, 2002.

(30) Foreign Application Priority Data
May 22, 2001 (GB) 0112494.0
Jul. 10, 2002 (GB) 0216027.3

(51) Int. Cl. A61K 31/519; A61P 25/00

(52) U.S. Cl. 514/252.16

(57) ABSTRACT

4-(2-Fluorophenyl)-6-methyl-2-(1-piperazinyl)thieno[2,3-D]pyrimidine or a salt thereof is useful for the treatment of pain.

### RELATED APPLICATIONS

This application is a continuation of U.S. application Ser. No. 10/617,847, filed Jul. 10, 2003, which is a continuation-in-part of International Patent Application No. PCT/GB02/02388, which designated the United States and was filed on May 21, 2002, published in English, which claims priority to Great Britain Application No. 0112494.0, filed May 22, 2001. This application also claims priority to GB 0216027.3, filed Jul. 10, 2002.

### FIELD OF THE INVENTION

This invention relates to a new therapeutic use for a known compound.

### BACKGROUND OF THE INVENTION

4-(2-Fluorophenyl)-6-methyl-2-(1-piperazinyl)thieno[2,3-D]pyrimidine monohydrate hydrochloride is known (see U.S. Pat. No. 4,695,568) and has shown activity as an antidepressant. It has serotonin and noradrenergic reuptake-blocking properties and these may be the mechanism of its action as an antidepressant. The compound also has 5HT-3 blocking activity.

Pain can be characterised as mild, moderate or severe. It can be acute or chronic in nature. Acute pain tends to resolve within minutes or days but if it persists beyond two weeks or so it is often termed chronic. Pain can be due to trauma or inflammation, and this is often termed nociceptive pain. However, pain not simply due to these causes but due mainly to nerve dysfunction or dysfunctional processing of sensory impulses is often referred to as neuropathic or neurogenic pain.

Acute nociceptive pain is well managed with non-steroidal anti-inflammatory drugs (NSAIDs) such as ibuprofen and aspirin if it is mild. For moderate pain, tramadol and codeine are useful. For severe pain, opiates such as morphine work well.

Mild to moderate chronic nociceptive pain due to inflammation is usually well managed with NSAIDs and COX-2 inhibitors although these drugs can cause serious gastric ulceration and bleeding. Tramadol is also very effective but can cause nausea, vomiting and constipation. For moderate nociceptive pain, opiates like oxycodone and codeine are useful but they cause constipation. For severe nociceptive pain, opiates such as morphine are the mainstay of treatment despite the risk of respiratory depression and addiction.

Fibromyalgia is a condition affecting 3-8 million people in the USA and approximately 80-90% of people affected are women. It is a chronic condition characterised by fatigue and widespread pain in muscles, ligaments and tendons. Previously the condition was known by other names such as fibrositis, chronic muscle pain syndrome, psychogenic rheumatism and tension myalgia.

Few adequate treatments exist for neuropathic pain, and only gabapentin is licensed for this purpose. New medicines are urgently required for neuropathic pain. There is also a need for safer and stronger analgesics for nociceptive pain.

Functional bowel disorders are very common and include irritable bowel syndrome (IBS) and functional dyspepsia. IBS is the most common disorder diagnosed by gastroenterologists and one of the more common encountered in general practice. The overall prevalence rate is similar (approx 10%) in most industrialised countries. Some estimates of prevalence have reached 20%. The illness has a large economic impact on health care use and indirect costs, chiefly through absenteeism.

IBS falls into two categories of equal prevalence, constipation-predominant and diarrhea-predominant. The available treatments are generally poor.

A recent approach to treating diarrhea-predominant IBS has involved the use of alosetron. This drug works by blocking the 5HT-3 receptor. Other drugs with this mechanism of action have shown some limited activity in this disease, including granisetron. Alosetron, although effective, was withdrawn due to side-effects on the colon.

A recent approach to treating constipation-predominant IBS involved agonising the 5HT4 receptor. Two such agonists are in clinical trials, i.e. tegaserod and prucalopride. Other approaches being explored include using 5HT1 agonists such as buspirone.

Functional dyspepsia is characterised by impaired accommodation of the stomach to a meal and epigastric pain discomfort or pain. There is often early satiety and weight loss. The disorder is not well understood. Treatments include antispasmodics and drugs affecting gut motility. Early studies suggest that buspirone and serotonin reuptake inhibitors may be useful.

### SUMMARY OF THE INVENTION

Surprisingly, it has been found that the known compound identified above (referred to herein as MCI-225) has activity in the treatment of pain and related conditions having a pain component, e.g. functional bowel disorder and fibromyalgia. Its combination of serotonin and noradrenergic reuptake blockade and 5HT-3 receptor blockade has not previously been identified as being responsible for activity in pain. It will be appreciated that any suitable form of the active principle may be used, e.g. another salt form, or a prodrug or active metabolite.

### DESCRIPTION OF THE INVENTION

By means of this invention, pain can be treated, e.g. controlled or prevented. Further, fibromyalgin and functional bowel disorders and associated pain symptoms can be treated, e.g. controlled or prevented. Such disorders include irritable bowel syndrome, including diarrhea-predominant, constipation-predominant, and alternating constipation/diarrhea IBS. The patient may be male or female, diarrhea-predominant IBS being particularly associated with women.

For use in the invention, the active compound can be formulated in any suitable manner together with a conventional diluent or carrier. The active compound is preferably administered by the oral route; other suitable routes of administration include sublingual/buccal, transdermal, intramuscular, intranasal, rectal, parenteral, subcutaneous, pulmonary and topical. An effective dose of the active agent will depend on the nature and degree of the complaint, the age and condition of the patient and other factors known to those skilled in the art. A typical daily dosage may be 0.1 mg to 1 or 5 mg.

A pharmaceutical composition containing the active ingredient may be in the form of a sublingual tablet or patch. Suitable compositions for oral use include tablets, troches, lozenges, aqueous or oily suspensions, dispersible powders or granules, emulsions, hard or soft capsules, syrups and elixirs. Suitable additives include sweetening agents, flavouring agents, colouring agents and preserving agents. Tablets contain the active ingredient in admixture with non-toxic pharmaceutically acceptable excipients, e.g. inert diluents such as calcium carbonate, sodium carbonate, lactose, calcium phosphate or sodium phosphate; granulating and disintegrating agents, for example corn starch, or alginic acid; binding agents, for example starch, gelatin or acacia, and lubricating agents, for example magnesium stearate, stearic acid or talc. The tablets may be uncoated or they may be coated by known techniques to delay disintegration and absorption in the gastrointestinal tract and thereby provide a sustained action over a longer period. For example, a time delay material such as glyceryl monostearate or glyceryl distearate may be employed. They may also be coated, to form osmotic therapeutic tablets for controlled release. Hard gelatin capsules may include an inert solid diluent, for example calcium carbonate, calcium phosphate or kaolin; soft gelatin capsules may include water or an oil medium, for example peanut oil, liquid paraffin or olive oil.

In the following studies, Study 1 shows the analgesic activity of MCI-225 at an oral dose of 30 mg/kg, in an in vivo model of inflammatory pain. The effect was comparable to that of indomethacin (1 mg/kg), an NSAID widely used for chronic pain such as arthritic pain.

In Study 2, using intact animals, the ability of a drug to inhibit the reflex depressor response to colorectal distension can be assessed. In this model, an inhibition of the reflex indicates modulation of visceral nociceptive neurotransmission and, therefore, the use of the drug in functional bowel disease (e.g IBS); see Kozlowski et al, 2000, Gut 46, 474-480. Allodynia and visceral pain are important components of functional bowel disease.

Study 1

Three groups of rats received vehicle, indomethacin or MCI-225. There were 13 rats in each group. Inflammatory pain was induced following a modified Randall Selitto method and the pain threshold of the inflamed paw was measured using a paw pressure analgesiometer. The threshold for paw withdrawal was measured in grams at 1 and 3 hours post dose. The results are shown in the following Tables.

| | | | Group mean (±sd) pain threshold (g) of inflamed paw at: | | | |
|---|---|---|---|---|---|---|
| | | Dose | 1 hour | 2 hour | 1 hour | 3 hour |
| Group | Treatment | (mg/kg) | pre-dose | pre-dose | post-dose | post-dose |
| 1 | Vehicle | 0 | 191.5 ± 88.56 | 146.5 ± 28.82 | 135.0 ± 36.23 | 135.0 ± 34.10 |
| 2 | MCI-225 | 30 | 147.7 ± 65.91 | 138.5 ± 34.72 | 170.8* ± 39.47 | 205.4** ± 68.30 |
| 3 | Indomethacin | 1 | 166.2 ± 68.32 | 144.2 ± 41.32 | 200.8* ± 82.96 | 210.0* ± 107.12 |

| | | | Group mean changes (±sd) in pain | |
|---|---|---|---|---|
| | | | threshold (g) from pre-dosereading at: | |
| | | Dose | 1 hour | 3 hour |
| Group | Treatment | (mg/kg) | post-dose | post-dose |
| 1 | Vehicle | 0 | −11.5 ± 50.56 | −11.5 ± 38.32 |
| 2 | MCI-225 | 30 | 32.3* ± 56.41 | 66.9** ± 65.85 |
| 3 | Indomethacin | 1 | 56.5** ± 56.18 | 65.8* ± 95.17 |

sd = Standard deviation
Statistical significance of difference from vehicle-treated group:
*p < 0.05,
**p < 0.01

The results show that MCI-225 was able to increase the pain threshold by 32.3 g at 1 hour and 66.9 g at 3 hours. There is a statistically significant difference between these values and those for vehicle at the same time intervals. On this basis, MCI-225 is useful for the treatment of inflammatory and other pain.

Study 2

Experiments were performed on male Sprague-Dawley rats (250-300 g). Anesthesia was induced with isoflurane (2.5% in oxygen) and maintained with alpha chlorolose (80 mg/kg i.v.). The left carotid artery was cannulated for the measurement of blood pressure and heart rate and the left jugular vein cannulated for drug administration. A tracheal cannula was implanted for artificial respiration if required. A 10 mm long latex balloon was inserted intrarectally so that the tip of the balloon was 20 mm from the anal verge (Kozlowski et al, supra). The balloon was connected via a double lumen cannula to a pressure transducer and also to a saline-filled syringe for inflation/deflation of the balloon. Throughout the experiment, body temperature was kept constant at 36-38 C using a homeothermic blanket.

Once stable baseline parameters were obtained (approximately after 20 minutes), the balloon was rapidly inflated with increasing volumes of saline (0.5-2.5 ml) for 30 seconds at 5 minute intervals, and the resultant change in blood pressure recorded. Three distinct response curves were constructed, with a 10 minute stabilisation period between each curve. In one group of animals, 10 minutes prior to the commencement of the final distension response curve, a single bolus of MCI-225 (3 mg/kg) was administered intravenously; in a second group of animals, a single bolus dose of vehicle was administered. The effect of MCI-225 and vehicle was determined by analysing the changes in colorectal distension that evoked depressor response.

Falls in arterial blood pressure (mean absolute decreases in mean arterial pressure in mmHg, with standard error of mean in brackets) evoked by distension of the balloon, before adding drug, at 0.5, 1.0, 1.5, 2.0 and 2.5 ml balloon volume were 2.7 (1.9), 12.4 (5.9), 24.0 (8.9), 36.3 (4.8) and 43.4 (6.0), respectively (all except final value n=6, final value n=5). Following administration of MCI-225 at 3 mg/kg i.v, the corresponding values were 2.2 (1.65), 6.3 (2.6), 10.6 (3.9), 15.3 (5.4) and 24.6 (7.3), respectively (all values except final value n=6, final value n=5).

The results clearly show that MCI-225 inhibited the distension-induced falls in blood pressure The falls in blood pressure evoked by 2.0 and 2.5 ml balloon volumes were reduced with statistical significance following administration of MCI-225 at 3 mg/kg, with p values (paired t test) of less than 0.01 and less than 0.05 respectively.

What is claimed is:

1. A method for the treatment of pain in a patient suffering therefrom, which comprises administering to the patient an effective amount of 4-2(2-fluorophenyl)-6-methyl-2-(1-piperazinyl)thieno[2,3-D]pyrimidine or a salt thereof.

2. The method according to claim 1, wherein the salt is the monohydrate hydrochloride.

3. The method according to claim 1, wherein the pain is nociceptive pain.

4. The method according to claim 1, wherein the pain is neuropathic pain.

5. A method for the treatment of fibromyalgia in a patient suffering therefrom, which comprises administering to the patient an effective amount of 4-(2-fluorophenyl)-6-methyl-2-(1-piperazinyl)thieno[2,3-D]pyrimidine or a salt thereof.

6. The method according to claim 5, wherein the salt is the monohydrate hydrochloride.
